(19) United States
(12) Patent Application Publication
(10) Pub. No.: US 2018/0177455 A1
(43) Pub. Date: Jun. 28, 2018

(54) DEVICE AND METHODS FOR MONITORING OR PREVENTING MISUSE OR ABUSE OF ANALGESICS

(71) Applicant: ISVIAL LLC, Irvington, NY (US)

(72) Inventors: Min Ding, Irvington, NY (US); Sabrina Shue, Irvington, NY (US)

(73) Assignee: ISVIAL LLC, Irvington, NY (US)

(21) Appl. No.: 15/822,468

(22) Filed: Nov. 27, 2017

Related U.S. Application Data
(60) Provisional application No. 62/426,548, filed on Nov. 27, 2016.
(60) Provisional application No. 62/428,422, filed on Nov. 30, 2016.
(60) Provisional application No. 62/556,645, filed on Sep. 11, 2017.

(51) Int. Cl. A61B 5/00; A61B 5/1455; A61B 5/0205

(52) U.S. Cl. CPC A61B 5/4845; A61B 5/14551; A61B 5/0022; A61B 5/02405; A61B 5/742; A61B 5/0205; A61B 5/746

(57) ABSTRACT

The invention relates to methods and devices for measuring or monitoring a subject undergoing one or more therapeutic treatments in real time to prevent drug abuse or misuse. The devices of the invention are intended to be worn or carried by the subjects, and they can thus be defined as portable or wearable devices. The devices are further configured to monitor the subjects and to prevent potential drug abuse or drug overdose.

### BACKGROUND OF INVENTION

Chemical analgesics have been widely used for pain management. They are highly effective in controlling pain, both acute and chronic. Over the past years, the use of analgesics in controlling acute and chronic pain has increased significantly. This is partially due to the increasingly large aging population both in the United States and foreign countries. For example, in 2016, there are 46 million people in the United States at the age of 65 or older. Many of these people suffer from chronic or acute pain, and require occasional or constant treatments. This trend expects to continue for the next several decades. The US population of 65 or older is expected to double by the year 2050.

While chemical analgesics provide highly effective pain relief, they may also have unintended and undesirable effects. Many of these analgesics when used incorrectly or abused could lead to serious side effects which could sometimes be fatal. This is especially true for certain class of analgesics, opioids or opioid analgesics. They are very effective in controlling pain. But at the same time, they are also highly addictive; over-use or misuse could lead to addiction, serious health side effects or even death when abused.

Over the past years, there have been numerous efforts in developing alternative therapies to the existing analgesics, especially opioids, in controlling chronic or acute pain via new mechanisms of action. In spite of these efforts and huge sum of capital expended to support these efforts, there has been very limited success. As a result, the current prevailing therapies in controlling pain are limited to several classes of chemical analgesics, in particular, opioids which function via suppression of mu receptor. Limited treatment options for controlling pain also leads to another unintended consequence, increasingly widespread addiction and misuse. Indeed, over the past ten years, pain drug abuse, misuse and even drug-related death have increased significantly.

In view of these issues, how to safely administer analgesics for pain control becomes an urgent issue. Even the US Food and Drug Administration has recently asked for industrial help in dealing with this issue. The present invention is directed to the development of means for real time monitoring of a subject which is undergoing analgesic treatment. It enables real time monitoring of a subject remotely. This also provides a mean to monitor, alarm and avoid potential drug misuse or abuse.

### BRIEF SUMMARY OF INVENTION

The invention generally relates to methods and devices for measuring or monitoring a subject undergoing therapeutic treatments in real time to prevent drug abuse or misuse. The devices of the invention are intended to be worn or carried by the subjects, and they can thus be defined as portable devices. The devices are further configured to monitor the subjects and to prevent potential drug abuse or drug overdose.

The devices provide real time measurement of a subject's physiologic conditions. Relevant measurements include, but are not limited to, such physiologic characteristics as heart rate, respiratory rate, pulse oxygen level, blood pressure, skin temperature, blood glucose level, liver function, renal function, and skin color.

The devices are configured to provide flexibility in the manner in which the data is collected, stored, displayed and transmitted. The use of such devices will allow a subject's physiologic characteristics to be monitored outside of a hospital or clinic, such as in a home or at work. For example, data on a subject's physiologic conditions collected by the device can be displayed in real time for review by the subject or other individual; data can be saved on the device for later download; and/or data can be transmitted wirelessly to a secondary device not worn or carried by the subject where it may be collected, stored, displayed and/or manipulated or used in some other manner.

The devices can be calibrated to produce an alarm to warn the subject that one or more of their physiologic conditions has drifted outside of a preset range, and/or to alert a medical professional, emergency medical personnel, or other individuals selected in advance. As the devices of the invention can also generate and transmit the physical location of the device, the monitoring service will be able to provide information related to the location of the subject which may be particularly important if the subject is incapacitated or unconscious.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1 is a diagram showing one embodiment of a device of the present invention.

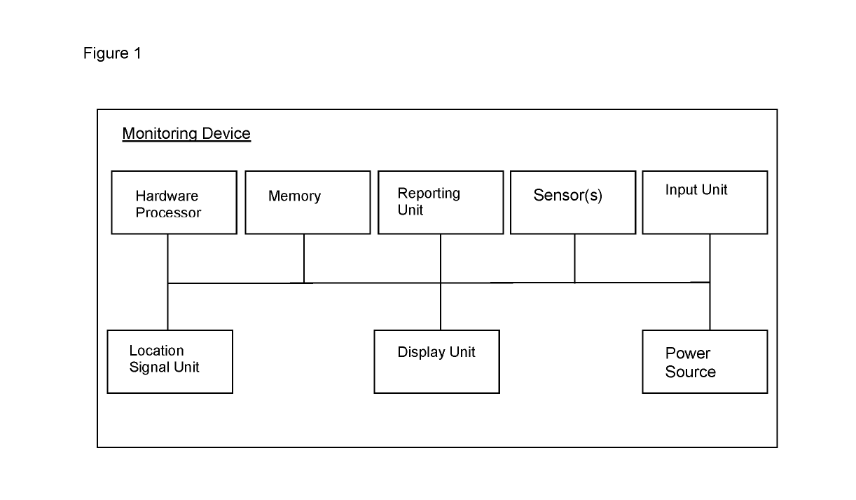

FIG. 2 is a flowchart showing the manner in which selected physiological conditions are monitored by sensors, correlated with pre-set levels, and reported.

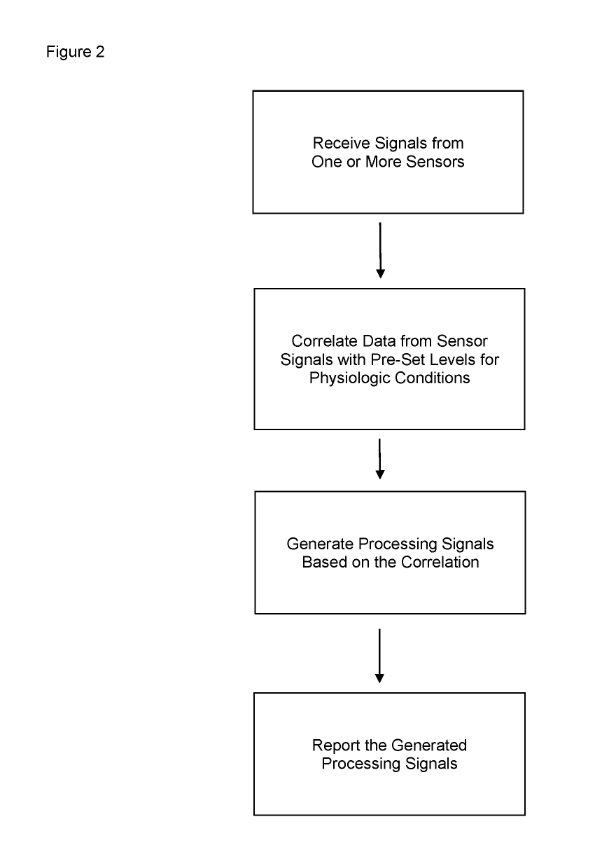

### DETAILED DESCRIPTION OF THE INVENTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +1-5-20% of the recited value) that one of ordinary skill in the art would consider equivalent to the recited value (e.g., having the same function or result). In some instances, the term “about” may include numerical values that are rounded to the nearest significant figure.

### II. The Present Invention

As detailed herein the present invention provides a method of monitoring a subject which is undergoing treatment or who is using certain drug(s), either therapeutic or recreational, in real time so as to prevent drug misuse, drug abuse or even drug-related death. The present invention further provides a device for monitoring or measuring one or more physiologic conditions of a subject in real time so as to prevent drug abuse or drug misuse.

### Device

The devices of the invention include each of the following components:
(i) one or more sensors, each sensor being configured to collect data representing selected physiologic conditions and generate a sensor signal based on the collected data;
(ii) a memory configured to store a program;
(iii) a hardware processor that executes the program and configured to:
   receive sensor signals from the one or more sensors,
   correlate the sensor signals with one or more pre-set levels corresponding to the selected physiologic conditions, and
   generate a processing signal when the correlation reveals the sensor signals deviate from the one or more pre-set levels by more than a selected percentage; and
(iv) a reporting unit configured to receive the processor signal from the hardware processor, and transmit the processing signal.

The one or more sensors are devices configured to collect data representing selected physiologic conditions and generate a sensor signal based on the collected data. For each physiologic condition, there may be a different sensor. The sensors may be configured to continuously collect the data representing selected physiologic conditions and generate a sensor signal based on the collected data, or configured to periodically collect the data representing selected physiologic conditions and generate a sensor signal based on the collected data. The periodic collection may be every 15, 30, 45 or 60 second for example, or every 5, 10, 15, 20, 25, 30 or more minutes.

The memory is configured to store a program and it may be non-transitory or transitory. The memory may also consist of a random access memory (RAM) and/or a non-volatile memory. Examples of memory suitable memories include, but are not limited to, a storage device, memory, unit, chip or circuit.

The hardware processor or controller executes the program and it is configured to receive sensor signals from the one or more sensors, correlate the sensor signals with one or more pre-set levels corresponding to the selected physiologic conditions, and generate a processing signal when the correlation reveals the sensor signals deviate from the one or more pre-set levels by more than a selected percentage. Such selected percentages include 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

The pre-set levels corresponding to the selected physiologic conditions may be determined from the subject's physiologic conditions prior to or at the time a course of medication administration begins in the subject, or at other times. Examples of a processor may be a circuit, chip, or other processing devices.

The reporting unit is configured to receive the processor signal from the hardware processor, and transmit the processing signal. Examples of reporting unit include, but are not limited to, a device that generates signals, such as an antenna.

The devices of the invention optionally include (v) a display unit configured to receive the processor signal and visually display values associated with the selected physiologic conditions. The display unit is configured to receive the processor signal and visually display values associated with the selected physiologic conditions or other associated signals. Examples of a display unit include, but are not limited to, a screen or alarm. The display unit may be further configured to generate an audible signal.

The devices of the invention optionally include (vi) a receiving unit configured to receive the processor signal and visually display values associated with the selected physiologic conditions. The receiving unit is configured to receive the processor signal and visually display values associated with the selected physiologic conditions, such as a receiver. In some aspects of the invention, the processing signal is wirelessly transmitted to a receiving unit configured to receive the processor signal and visually display values associated with the selected physiologic conditions. The receiving unit may be further configured to generate an audible signal.

The devices of the invention optionally include (vii) a location signal unit configured to generate and transmit the physical location of the device. In some aspects, the location signal unit generates and transmits a GPS or a cellular signal.

The devices of the invention optionally include (viii) an input unit physically or wirelessly connected to the hardware processor and configured to program or adjust the pre-set levels corresponding to the selected physiologic conditions. Input unit may be a keyboard, screen which is built-in, or a computer, screen, keyboard, or another device which is connected via wire or wireless to the devices of the invention.

The devices of the invention optionally include (ix) a power source which provides power for the devices. Power source can be an outlet and preferably a battery which is portable and can be carried around.

The devices of the invention optionally include (x) a switch which may be automatically or manually turned on or off. Sometimes, the switch may be controlled remotely. In some other examples, the switch may be controlled by a timer so that the device is turned on or off at periodically or at a defined time.

The devices of the invention may be portable or wearable by the subject. For example, the devices of the invention may in the form of a watch, ring, wrist band, arm or leg band. Alternatively, the devices of the invention may be strapped to an area of the body of the subject, such as to the chest or back, forehead or neck, or some other area.

### Physiologic Conditions

As indicated herein, the devices of the invention provide real time data on a subject's physiologic conditions. Relevant physiologic conditions include, but are not limited to, such physiologic characteristics as heart rate, respiratory rate, pulse oxygen level, blood pressure, skin temperature, blood glucose level, liver function, renal function, and skin color. One of the preferred embodiments is the measurement of respiratory rate and/or pulse oxygen level. In certain embodiments, the device monitors and/or measures 1, 2, 3, 4, 5, 6, 7, 8 or all 9 of these physiologic conditions. In a particular embodiment, the device monitors and/or measures one or more of heart rate, respiratory rate, pulse oxygen level, blood pressure, or two or more of these physiologic conditions, or three or more of these physiologic conditions, or all four of these physiologic conditions.

With respect to each of the physiologic conditions discussed herein, values in the data collected from a subject will vary by such factors as the age, sex, weight, height, and medical condition of the subject. Further, the measurements can vary depending on whether the subject is at rest or engaged in some activity, exercise or exertion. Accordingly, one embodiment of the invention is a device or method that is automatically or manually adjusted based on the subject's conditions.

For the sake of consistency, the specific parameters discussed herein for the noted physiologic conditions are with respect to a subject at rest. It will be apparent that these parameters may be adjusted based on the activity level of a particular subject (e.g. the monitoring of the physiologic conditions of an athlete engaged in an exercise, sport, event, etc.). Accordingly, one embodiment of the devices of the invention is to allow the pre-set range to be disabled or changed manually by either a doctor or a patient/subject based on different conditions, including the subject's normal baseline and the health or activity level of the subject.

In another embodiment, the devices of the invention may optionally contain a program which adjusts the pre-set range while the subject is not at rest. For example, when a subject is exercising, the heart rate of the subject may increase. As a result, one or more other pre-specified parameters may be adjusted or increased at times in proportion to the increase in heart rate.

### Base Level

As detailed in the paragraphs below, data collected from sensors of the devices of the invention is correlated with a base level corresponding to the same physiologic condition(s) of the subject. For each physiologic condition being monitored, there should be a corresponding base level. Base level can be a specific value or a range. For example, base level of a subject's heart rate may be 60 beats per minute or 60-80 beats per minute.

The base level may be determined based on the standard or range generally observed in a normal, healthy subject or observed in a subject having the same or similar disease or condition. For example, a normal healthy person's heart rate may be in the range of 60-90 beats per minute.

More preferably, the base level should be determined or established based on the subject's specific health and condition. For example, one preferred embodiment of the invention is to detect and measure a subject's physiologic conditions prior to, concurrently with, or immediately after administration to the subject of the treatment or a drug as described herein and use of the measurement as the base level or base range. One advantage of using such measures as the base level or range is to allow such pre-set level to be within a relatively narrow range. The base level of a specific physiologic condition may be a specific value and may also be a range.

### Pre-Set Level or Range

Generally, a subject's physiologic condition may change and fluctuate within a certain range. As long as such variation or change is within a certain range, it should not affect the subject's physical health or the subject's normal or daily function. The devices of the invention are programmed to contain a pre-set range allowable for one or more physiologic condition specified herein. Alternatively, the devices of the invention may be programmed to contain a pre-set range that are not allowable for one or more physiologic conditions.

Such pre-set range may be specified by the subject, a third party, or preferably a treating physician. The pre-set range may be established based on each individual physiologic condition. For example, it may be established based on what is observed in a normal healthy subject or in a subject having the same or similar health or disease conditions.

More preferably, the pre-set range may be established based on the subject's specific physiologic condition or the base level as measured or established above. For a specific physiologic condition, the pre-set rage may be within about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of the base level, either higher or lower or with both higher and lower ranges. For example, when the subject's heart rate immediately prior to the treatment is 70, the pre-set level may be in the range of no more than 15%-20% higher, or no more than about 15%-20% lower, or with both higher and lower range.

It should be noted that the pre-set range based on the base level may have a higher level and lower level. Such higher level and lower level need not be identical percentage. For example, a pre-set range for a subject's renal function may be 15% higher and 10% lower of the base level as measured in a subject.

For certain physiologic conditions, percentage increase or decrease may not be the best description. For example, decrease or increase in a subject's heart rate may be best measured by the number of heart beats per minute. Oxygen level may be best described by the change of percentage of oxygen level in blood, e.g. from 98% to 93% oxygen level. For those physiologic conditions, the pre-set range described above may be rounded to the closest number. Some of them may be described in more details below for each of these physiologic conditions.

Alternatively, in some devices, the pre-set range may be a range that falls outside what is described above. For example, the pre-set range may be heart range of above 100 beats per minute or below 40 beats per minute. One embodiment of the present invention is an input device or option that allows the subject, a third party, preferably a treating physician, to adjust the pre-set range for one or more physiologic conditions.

The pre-set range may be stored on the devices. Sometime, it is also possible to store such pre-set range in a processor that is located remotely, as long as such processor can easily communicate with the device via wire or wireless.

### Monitoring Process

During the active monitoring, one or more of a subject's physiologic conditions are measured or monitored in real time. In some embodiments, such measurement or monitor may be performed on a continuous basis. In some other embodiments, such measurement or monitor may be performed periodically, for example, in about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 or more seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more minutes, or 1, 2, 3, 4, 5, 6, 7, 8, 9, 10, 11 or 12 or more hours.

For each physiologic condition, the real-time measurement or monitoring may be programmed differently. In some embodiments, the devices of the invention may be programmed to monitor or measure one or more physiologic conditions on a continuous basis while measuring or monitoring other physiologic conditions periodically. In some embodiments, the devices of the invention may be programmed to monitor or measure one or more physiologic conditions every 30-60 seconds while measuring or monitoring other physiologic conditions every 5-10 minutes.

The devices of the invention are programmed that, when the measurement of a subject's one or more physiologic conditions fall within or outside the pre-set range, a signal may be generated that is sent to a display unit for review by the subject and/or receiving unit for review by a third party, such as a doctor or monitoring service. For example, if the pre-set level of blood oxygen level is at or above about 94% (blood oxygen level), a signal or alarm is triggered when oxygen level as measured in a subject falls below this level. In another example, if the pre-set level is at or below 40 heart beats per minute, a signal may be sent if a subject's heart rate falls below 40 beats per minute.

Sometimes, instead of or in addition to sending a signal to a display unit, the device may trigger an alarm, such as audio, video, vibration or signals in other ways so as to alert the subject or to alert the third party.

In some embodiments, the devices of the invention may be programmed so that the signal is only generated when deviations occur for two or more physiologic conditions or upon combinations of different physiologic conditions. For example, a device monitoring a subject undergoing treatment of an opioid analgesic may send out an alarm when the heart rate is of 10% below the base level and when the pulse oxygen level at or below about 93%.

In some embodiments, the devices of the invention may be programmed so that the signal is only generated when deviations occur for a certain period of time, such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 or more seconds, or 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 or more minutes.

In some embodiments, the devices of the invention may be programmed so that measurement of some physiologic conditions is turned off or dormant. Such measurement is only turned on when other physiologic condition or conditions fall within or outside a certain pre-set range. For example, liver or renal functions may not be constantly measured. Only when the heart rate falls outside a pre-set range will the measurement of oxygen level takes place to determine whether or not there is any deviation from the pre-set range.

### Heart Rate

With respect to heart rate, normal health subjects will typically have about 60-90 beats per minute, although variation from this range by up to 10%, from either end of the range, may also be acceptable. Accordingly, a general pre-set level without taking into account an individual subject's condition may be from about 50-100 beats per minute.

More preferred embodiment is a pre-set level based on a subject's base level or his/her normal heart rate. This may be obtained based on the subject's medical or health history. It may also be based on a subject's heart before, concurrently or immediately after the need for the monitoring services or when a drug is administered. It is also possible to obtain the average heart rate that a subject may have over a certain period of time, such as over one hour, one day, one week, or month internal, and use it as the base level or pre-set level.

A pre-set heart rate may be range that, compared to a base level, has a higher, lower or both heart beats per minute differing by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 or more beats per minute. Optionally, the pre-rate levels may contain two or more levels, e.g. a lower level and a higher level. Such lower level and higher level may deviate from the base by different numbers of heart beats per minute.

Sensors that may be used in the devices of the invention to collect data representing heart rate include, but are not limited to, a heart rate monitor. There are various types of heart rate sensing devices or sensors available that may be used in the present invention using radio signals or microprocessor, or other mechanism of actions.

### Respiratory Rate

With respect to respiratory rate, the normal respiratory rate for a normal person is about 10-40, specifically about 5-10, 10-12, 12-15, 15-17, 17-20, 20-30, or 30-40 breaths per minute, although the base level for each subject could vary significantly based on a subject's physical health and condition. For example, a subject suffering from COPD or other pulmonary diseases may have a much higher respiratory rate at the base level. The rate also differs based on age. For example, normal base level for a newborn may be from 30-40 breaths per minute. A child below 18 year old may be 15-40.

A pre-set range of a subject's respiratory rate may differ from the base level, or the standard respiratory rate for a normal person if that is the base level used, by about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 or more breaths per minute.

Sensors that may be used in the devices of the invention to collect data representing respiratory rate include, but are not limited to, a pneumograph and a capnography. Additional sensors may include electrocardiogram, photoplethysmogram, accelerometry, and other sensors that may detect or measure a subject's respiratory rate.

### Oxygen Level

One of the preferred physiologic conditions to be used in the present invention is a subject's oxygen level, preferably pulse oxygen. The oxygen level is generally measured by the level of oxygen in the blood, preferably in arterial oxygen. Sometime, the pulse oxygen level as measured by oximeter is measured by the percentage of oxygen in blood.

For a normal subject, the oxygen level may be in the range of 75 to 100 millimeters of mercury (mm Hg). However, this level should be adjusted based on each subject and state of health of the subject. As measured by pulse oximeter, the base level may be from about 95%-100% or more.

With respect to the oxygen level, the pre-set level, as measured by pulse oximeter, may be in the range of a pulse oxygen level of less than about 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70% or less.

In another embodiment, the pre-set oxygen level may be established based on a subject's specific normal oxygen level preferably measured over a certain period of time. The pre-set level may be at or below about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 65%, 60%, 55%, 50%, 45%, 40%, 35% or less of the base oxygen level.

Sensors that may be used in the devices of the invention to collect data representing pulse oxygen level include, but are not limited to, a pulse oximeter or other similar devices.

### Blood Pressure

Blood pressure is generally measured by systolic and diastolic blood pressure. For a normal subject, the base level systolic blood pressure may be about 90 to 135 and diastolic blood pressure may be about 60-90 as measured by mm Hg. However, it should be noted that the blood pressure varies greatly from subject to subject.

With respect to blood pressure, the pre-set level will be based on the standard systolic blood pressure, or diastolic blood pressure or both, observed in a normal subject. For example, the pre-set level of systolic pressure may be at or above about 110-120, 120-130, 130-140, 140-150, 150-160, 160-170 mm Hg, or at or below about 80-90, 70-80, 60-70, 50-60 mm Hg. For diastolic blood pressure, the pre-set level may be at or above about 80-90, 90-100, 100-110 mm Hg, or at or below about 60-70, 50-60, 40-50, 30-40, 20-30 mm Hg. It should be noted that, for each of systolic blood pressure and diastolic blood pressure, there may be a higher range and lower range representing hypotension or hypertension.

More preferably, the pre-set level may be established based on a subject's normal blood pressure. For example, the pre-set level for systolic blood pressure, diastolic blood pressure or both may be in the range of either about or below 95%, 90%, 85%, 80%, 75%, 70%, 65%, 60%, 55%, 50%, 45%, 40%, 35%, 30%, 25%, 20% or 15% of a subject's normal blood pressure. Alternatively or in addition, the pre-set level for systolic blood pressure, diastolic blood pressure or both may be in the range of either about or above 105%, 110%, 115%, 120%, 125%, 130%, 135%, 140%, 145%, 150%, 155%, 160%, 165%, 170%, 175%, 180%, 185%, 190%, 195%, or 200% of a subject's normal blood pressure.

Sensors that may be used in the devices of the invention to collect data representing blood pressure include, but are not limited to, a sphygmomanometer.

### Skin Temperature

With respect to skin temperature, the base level for a normal subject often falls with the range of about 36 to 38° C. although this may vary based on a subject's health condition, age and other conditions.

The pre-set level may be below or above the lower range or higher range of a base level, such as a based level of 36 to 38° C., by about 0.1° C., 0.2° C., 0.3° C., 0.4° C., 0.5° C., 0.6° C., 0.7° C., 0.8° C., 0.9° C., 1° C., 1.1° C., 1.2° C., 1.3° C., 1.4° C., 1.5° C., 1.6° C., 1.7° C., 1.8° C., 1.9° C., 2.0° C., 2.1° C., 2.2° C., 2.3° C., 2.4° C., 2.5° C., 2.6° C., 2.7° C., 2.8° C., 2.9° C., 3.0° C., 3.1° C., 3.2° C., 3.3° C., 3.4° C., 3.5° C., 3.6° C., 3.7° C., 3.8° C., 3.9° C., 4.0° C., 4.1° C., 4.2° C., 4.3° C., 4.4° C., 4.5° C., 5.0° C., 5.5° C., 6.0° C., 6.5° C., 7.0° C., 8.0° C., 9.0° C., 10.0° C., 11.0° C., 12.0° C., 13.0° C., 14.0° C., 15.0° C., 16.0° C., 17.0° C., 18.0° C., 19.0° C. or 20.0° C.

The pre-set level may also be calculated based on the higher and a lower percent of the base level. For example, a pre-set level may be at or below about 95%, 90%, 85%, 80%, 75%, 70%, 65%, 60% or at or above about 105%, 110%, 115%, 120%, 125%, 130%, 135%, 140%, 145% or 150% of the base level.

Sensors that may be used in the devices of the invention to collect data representing skin temperature include, but are not limited to, a thermometer or other similar devices.

### Blood Glucose Level

Another physiologic condition is the blood glucose level. With respect to blood glucose level, the base level for a normal subject will typically be a blood glucose level of about 70-100 mg/dL prior to food intake (fasting). This base level varies significantly because of food intake and health condition of a subject. For example, a subject who suffers from diabetes may have a much higher blood glucose level in the range of 90-130 mg/dL.

Based on the standard range, the pre-set level could have a lower range of at or below about 65, 60, 55, 50, 45, 40, 35, 30, 25, 20 mg/dL and could have a higher range of at or above about 105, 110, 115, 120, 125, 130, 135, 140, 145, 150, 160, 170, 180, 190, 200, 210, 220, 230, 240, 250, 260, 270, 280, 290 or 300 mg/dL.

More preferably, the pre-set level may be based on a subject's normal glucose level measured preferably over a certain period of time. The pre-set let may be below such base level by about 5, 10, 15, 20, 25, 30, 35, 40, 45, 50, 60, 65, 70, 75 or 80 mg/dL or less. It may also be above such base level by about 5, 10, 15, 20, 25, 30, 35, 40, 45, 50, 60, 65, 70, 75, 80, 85, 90, 95, 100, 105, 110, 115, 120, 125, 130, 135, 140, 145, 150, 155, 160, 165, 170, 175, 180, 185 mg/dL or more.

Sensors that may be used in the devices of the invention to collect data representing blood glucose level include, but are not limited to, a blood glucose monitor device.

### Liver Function

Another physiologic condition is the liver function. There are several ways to monitor the liver function. Most common methods are to monitor and measure certain liver enzyme activities, including measurement of blood bilirubin concentration, blood level of albumin concentration, blood alkaline phosphatase (ALP), blood aspartate aminotransferase (AST) and blood alanine aminotransferase (ALT). For the purpose of this invention, one, two, three, four or all five of these may be monitored.

With respect to liver function, the base level for blood bilirubin in a normal subject is in the range of 0.3-1 mg/dL. In an older subject, the base level may be lower from 0-1 mg/dL. The pre-set level may be at or above about 1, 1.1, 1.2, 1.3, 1.4. 1.5. 1.6. 1.7. 1.8. 1.9. 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4 mg/dL. Another way of defining the pre-set level is based on the percentage difference which is at or above 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 320%, 340%, 360%, 380% or 400% of the base level.

The base level of albumin level is in the range of 3.5 to 5.4 g/dL in a normal subject. The pre-set level could be either lower, higher or both than the base level range. The pre-set level could be at or below about 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, 0.1 g/dL. It could also be at or above 5.4, 6, 6.5, 7, 7.5, 8, 8.5, 9, 9.5, 10, 11, 12, 13, 14, 15, 16, 17, 18, 19 or 20 g/dL.

The base level for blood alkaline phosphatase (ALP) concentration in a normal subject should be in the range of about 40-115 U/L. The base level for blood aspartate aminotransferase (AST) concentration should be in the range of 10-40 U/L. The base level for blood alanine aminotransferase (ALT) concentration may in the range of about 9-46 U/L. The pre-set level may be at or above about 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 320%, 340%, 360%, 380% or 400% of the base level. Alternatively or in addition, the pre-set level may also be at or below about 90%, 80%, 70%, 60%, 50%, 40%, 30%, 20%, or 10% of the base level.

Sensors that may be used in the devices of the invention to collect data representing liver function include, but are not limited to, a portable liver functional testing device,

### Skin Color

Another physiologic condition is the change in skin color. Drastic change in skin color without excess exposure to sunlight may indicate change in a subject's condition, such as liver function. Therefore, another physiologic condition of the invention is the measurement and monitoring of change in skin color. Because each subject may have a different skin color, the base level should be measured based on individual subjects. For example, the base level of a subject's skin color may be monitored or measured prior to, concurrently with, or immediately after the subject is administered with a treatment, such as opioid analgesic.

The pre-set level may be the percentage change of the skin color, such as darkening or lightening. For example, the pre-set level may be at or above about 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 320%, 340%, 360%, 380% or 400% of the base level. Alternatively or in addition, the pre-set level may also be at or below about 90%, 80%, 70%, 60%, 50%, 40%, 30%, 20%, or 10% of the base level.

Sensors that may be used in the devices of the invention to collect data representing skin color include, but are not limited to, skin color scanners or other similar devices.

### Renal Function

Another physiologic condition is the renal function. Common measurement of renal function includes, without limitation, the measurement of blood urea concentration and blood creatinine concentration. In a normal subject, the base level of blood urea concentration is about 7-25 mg/dL and the base level of blood creatinine concentration is about 0.6-1.35 mg/dL.

The pre-set level may be at or above about 100%, 105%, 110%, 115%, 120%, 125%, 130%, 135%, 140%, 145%, 150%, 160%, 170%, 180%, 190%, 200%, 210%, 220%, 230%, 240%, 250%, 260%, 270%, 280%, 290%, 300%, 320%, 340%, 360%, 380% or 400% of the base level. Alternatively or in addition, the pre-set level may also be at or below about 90%, 80%, 70%, 60%, 50%, 40%, 30%, 20%, or 10% of the base level.

Sensors that may be used in the devices of the invention to collect data representing renal function include, but are not limited to, a portable renal function analyzing device.

### Subjects

The subjects that may be outfitted with the devices of the invention will typically be a human. However, as used herein a subject may also be a non-human primate, bird, horse, cow, goat, sheep, a companion animal, such as a dog, cat or rodent, or other mammals.

The subject may be male or female, and may be of any age. Preferably, the subjects may be in the age of 30, 35, 40, 45, 50, 55, 60, 65, 70, 75, 80, 85 or older, or in the age of 10, 9, 8, 7, 6, 5, 4, 3, 2, 1 or younger.

The subject need only be one for whom the monitoring or measuring is desired. Thus, while relevant subjects may often be a patient taking a medication or having a medical condition, the subject may be an athlete, for example, or simply a person who desires to have knowledge regarding one or more of their physiologic conditions.

When the subject is a patient taking medication, the medication will typically be one where there is a risk of adverse reaction or overdose. Such medications include, but are not limited to, analgesic such as a narcotic or opioid analgesic, a benzodiazepine, and other pain relievers, such as mu receptor antagonist/agonist.

Relevant narcotics and opioids include, but are not limited to, codeine, oxycodone, hydrocodone, dilaudid, fentanyl, morphine sulfate, tapentadol, methadonebuprenorphine, meperidine, oxymrophone, tramadol, and levorphanol, and any combination thereof. Relevant benzodiazepines include, but are not limited to, alprazolam, chlordiazepoxide, cloazepam, clorazepate, diazepam, estazolam, flurazepam, lorazepam, oxazepam, temazepam, and triazolam, and any combination thereof.

When the subject is a patient having a medical condition, the medical condition will typically be one where data regarding physiologic conditions is relevant to a diagnosis or to the continuing care being provided by a doctor. Such patients may be taking a course of medication, or have a condition for which it is important to monitor physiologic conditions. Such patients may have an addition to a particular medication or an illicit substance. Relevant illicit substances include, but are not limited to, heroin, cocaine, and methamphetamine.

### Methods

Another aspect of the invention is the methods of monitoring a subject undergoing drug treatment or who is at risk of drug overdosing or drug abuse. More specifically, the invention describes a method of monitoring a subject in need thereof, comprising measuring or monitoring one or more of the subject's physiologic conditions selected from a group consisting of heart rate, respiratory rate, pulse oxygen level, blood pressure, skin temperature, blood glucose level, liver function, renal function, and skin color in real time, comparing such measurement with a pre-set level or range, generating a signal in the event that the measurement exceeds or falls within such pre-set level or range.

One embodiment of the invention is a method of monitoring a subject who is receiving a medical treatment, comprising measuring or monitoring one or more of the subject's physiologic conditions selected from a group consisting of heart rate, respiratory rate, pulse oxygen level, blood pressure, skin temperature, blood glucose level, liver function, renal function, and skin color, comparing such measurement with a pre-set range, generating a signal in the event that the measurement exceeds or falls within such pre-set level range, and locating the subject.

Another embodiment is a method of monitoring a subject undergoing medical treatment of one or more analgesics in real time, comprising measuring one or more the subject's physiologic conditions selected from a group including heart rate, respiratory rate, pulse oxygen level, blood pressure, skin temperature, blood glucose level, liver function, renal function, and skin color before, concurrently with, or soon after the subject receives the medical treatment, establishing a base level for one or more of these physiologic conditions, establishing a pre-set level of range for one or more of these physiologic conditions, measuring or monitoring the subject for the selected physiologic condition(s), and generating a signal in the event that the real-time measurement of the selected physiologic condition(s) falls within or falls outside the pre-set level or range.

Yet another embodiment is a method of monitoring a subject undergoing treatment with one or more analgesics or at risk of taking one or more analgesics, comprising (1) measuring in real time one or more the subject's physiologic conditions selected from a group including heart rate, respiratory rate, pulse oxygen level, blood pressure, skin temperature, blood glucose level, liver function, renal function, and skin color in real time using one or multiple sensors, (2) transmitting one or more signals from the sensor(s) to one or more processors which correlate the sensor signals with one or more pre-set levels corresponding to the selected physiologic conditions and which generates a processing signal when the correlation reveals the sensor signals fall outside or within the pre-set levels, (3) transmitting a signal from the processor to a reporting unit when the real time measurements of one or more of the selected physiologic conditions falls within or falls outside the pre-set levels for the corresponding physiologic condition(s), (4) generating a signal from the reporting unit so that the subject, a third party or multiple parties may be notified.

Another embodiment of the invention is a method of monitoring a subject undergoing medical treatment of one or more analgesics, comprising measuring or monitoring one or more of the subject's physiologic conditions selected from a group including heart rate, respiratory rate, pulse oxygen level, blood pressure, skin temperature, blood glucose level, liver function, renal function, and skin color, comparing such measurement with a pre-set level or range, generating a signal in the event that the measurement exceeds or falls within such pre-set range.

Yet another embodiment of the invention is a method of monitoring a subject undergoing medical treatment of one or more opioid analgesics, comprising measuring or monitoring one or more of the subject's physiologic conditions selected from a group consisting of heart rate and pulse oxygen level, comparing such measurement with a pre-set range, generating a signal in the event that the measurement exceeds such pre-set range.

As described herein, the opioid analgesic may be selected from a group consisting of codeine, oxycodone, hydrocodone, dilaudid, fentanyl, morphine sulfate, tapentadol, methadonebuprenorphine, meperidine, oxymrophone, tramadol, levorphanol, or any combination thereof. Other medicament includes benzodiazepines, such as alprazolam, chlordiazepoxide, cloazepam, clorazepate, diazepam, estazolam, flurazepam, lorazepam, oxazepam, temazepam, triazolam, and any combination thereof.

The methods of the present invention may further optionally comprise a step of generating a signal so that a third party, such as a caretaker, physician or others, may be able to determine the location of the subject remotely. For example, the device of the invention may also be used as a GPS locator or tracking device so that the subject may be located.

The methods of the invention may also be used to identify and/or prevent drug abuse or mis-use in a subject in need thereof. Such drug could be an analgesic and it could also be an illicit or recreational drug, such as cocaine or heroin.

The devices of the present invention may be outfitted to a subject in need thereof.

While the invention has been described with reference to certain particular embodiments thereof, those skilled in the art will appreciate that various modifications may be made without departing from the spirit and scope of the invention. The scope of the appended claims is not to be limited to the specific embodiments described.

1-52. (canceled)

53: A method for remotely monitoring misuse or overdose of an opioid analgesic in a subject in need thereof, comprising
(a) measuring the subject's pulse oxygen level prior to, currently or immediately after administration of the opioid analgesic in the subject to establish a baseline level using a sensor in a wireless and wearable device,
(b) storing the baseline level in a memory contained in the device,
(c) establishing a pre-set range based on the baseline level using a hardware processor in the device,
(d) monitoring in real-time the subject's pulse oxygen level at a frequency of no more than 5 minutes for each measurement using the sensor,
(e) comparing the real-time measurement of the subject's pulse oxygen level with the pre-set range using a hardware processor, and
(f) generating an electronic signal to be transmitted to a remote location when the real-time measurement of the subject's pulse oxygen level exceeds the pre-set range.

54: The method of claim 53, further comprising the step of generating vibration or an audible signal to alert the subject once the real-time measurement of the subject's pulse oxygen level exceeds the pre-set range.

55: The method of claim 53, further comprising a step of monitoring the subject at an increased frequency once the subject's pulse oxygen level reaches a certain level.

56: The method of claim 53, wherein the pre-set range is at or above 10% of the baseline level.

57: The method of claim 53, wherein the device further comprises a location signal unit configured to generate and transmit the physical location of the device.

58: The method of claim 57, wherein the location signal unit generates and transmits a GPS or a cellular signal.

59: The method of claim 53, wherein the wireless and wearable device is a watch, wristband, or ring.

60: The method of claim 53, wherein the opioid analgesic is selected from a group consisting of codeine, oxycodone, hydrocodone, dilaudid, fentanyl, morphine sulfate, tapentadol, methadonebuprenorphine, meperidine, oxymrophone, tramadol, levorphanol, heroin, cocaine, and methamphetamine, any of their salt or ester form, and any combination thereof.

61: The method of claim 53, wherein the pre-set level is at or above 12% below the baseline level.

62: The method of claim 53, wherein the pre-set level is at or above 15% below the baseline level.

63: The method of claim 53, further comprising measuring the subject's heart rate in real time.

64: The method of claim 53, wherein the device further comprises a display device.

65: A method for remotely monitoring misuse or overdose of an opioid analgesic in a subject in need thereof, comprising
(a) measuring the subject's physiological conditions of pulse oxygen level and heart rate prior to, currently or immediately after administration of the opioid analgesic in the subject using sensors in a wireless and wearable device to establish a baseline level for each of these physiological conditions,
(b) storing the baseline levels in a memory contained in the device,
(c) establishing a pre-set range based on the baseline level for each of these physiological conditions using a hardware processor in the device,
(d) monitoring in real-time the subject's pulse oxygen level at a frequency of no more than 5 minutes for each measurement,
(e) monitoring in real-time the subject's heart rate,
(f) comparing the real-time measurement of the subject's physiological conditions with the pre-set ranges using a hardware processor, and
(g) generating an electronic signal to be transmitted to a remote location when the real-time measurement of either or both of the subject's physiological conditions exceed the pre-set range.

66: The method of claim 65, wherein the device further comprises a location signal unit configured to generate and transmit the physical location of the device.

67: The method of claim 66, wherein the location signal unit generates and transmits a GPS or a cellular signal.

68: The method of claim 65, wherein the wireless and wearable device is a watch, wristband, or ring.

69. The method of claim 65, wherein the opioid analgesic is selected from a group consisting of codeine, oxycodone, hydrocodone, dilaudid, fentanyl, morphine sulfate, tapentadol, methadonebuprenorphine, meperidine, oxymrophone, tramadol, levorphanol, heroin, cocaine, and methamphetamine, any of their salt or ester form, and any combination thereof.

70: The method of claim 65, wherein the subject suffers from or is susceptible to opioid addiction.

71: The method of claim 65, further comprising the step of generating vibration or an audible signal.

72: A method for remotely monitoring misuse or overdose of an opioid analgesic in a subject in need thereof, comprising
(a) measuring the subject's physiological conditions of pulse oxygen level and heart rate prior to, currently or immediately after administration of the opioid analgesic in the subject using sensors in a wireless and wearable device to establish a baseline level for each of these physiological conditions,
(b) storing the baseline levels in a memory contained in the device,
(c) establishing a pre-set range based on the baseline level for each of these physiological conditions using a hardware processor in the device,
(d) monitoring in real-time the subject's pulse oxygen level at a frequency of no more than 2 minutes for each measurement,
(e) monitoring in real-time the subject's heart rate,
(f) comparing the real-time measurement of the subject's pulse oxygen level with the pre-set range using a hardware processor,
(g) generating an electronic signal to be transmitted to a remote location when the real-time measurement of either or both of the subject's physiological conditions exceed the pre-set range, and
wherein the wireless and wearable device is a watch, ring or wristband and an opioid analgesic is selected from a group consisting of codeine, oxycodone, hydrocodone, dilaudid, fentanyl, morphine sulfate, tapentadol, methadonebuprenorphine, meperidine, oxymrophone, tramadol, levorphanol, heroin, cocaine, and methamphetamine, any of their salt or ester form, and any combination thereof.
